(19) United States
(12) Patent Application Publication
(10) Pub. No.: US 2009/0199856 A1
(43) Pub. Date: Aug. 13, 2009

(54) Protective garment for masks

(76) Inventor: Marcell Berlin, Chimacum, WA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/069,347

(22) Filed: Feb. 7, 2008

(51) Int. Cl. A62B 7/00; A61B 19/00; A62B 18/02

(52) U.S. Cl. 128/206.13; 128/898; 128/206.21

(57) ABSTRACT

A protective garment adapted to be worn by a patient fitted with mask having a strap. The garment is constructed from a soft, resilient material, such as yarn which is knitted, or foam. The garment includes (1) an elongate central portion having a first end and an opposite second end. The central portion has a recessed region and adjacent peripheral raised regions. The recessed region and peripheral raised regions extend substantially the entire length of the central portion. The recessed region receives the strap of the mask. The garment further includes cheek pads integral with the first and second ends of the central portion. The protective garment prevents the face, ears and head of the patient from chafing caused by direct contact between the patient's skin and the mask and strap.

### BACKGROUND

This invention relates generally to the field of medical apparatus and more particularly to a protective garment which is worn on the head and face of a patient to protect the patient's skin from chafing when they wear a mask used to deliver oxygen or an anesthetic.

Critically ill patients, the elderly and even children are sometimes required to wear an oxygen mask to assist in breathing. Often, the patient is unconscious. Sometimes the mask is worn for extended periods of time. The mask, and its associated strap which holds the mask on the patient's face, is a source of chafing of the patient's skin, particularly in the cheeks, around the ears, and the back of the head. This invention satisfies a long-felt need in the art for a protective garment for patients to wear which prevents such chafing.

### SUMMARY

In a first aspect, a mask protector is described which is adapted to be worn by a patient fitted with mask, e.g., an oxygen supply mask. The mask has an elastic strap for enabling the mask to be held on the patient's head. The mask protector is in the form of a protective garment constructed from a soft, resilient material. The garment includes (1) an elongate central portion and (2) first and second cheek pads integral with the central portion and provided at opposites ends of the central portion. The central portion has a recessed region (e.g., channel) and peripheral raised regions on either side of the recessed region. The recessed region and peripheral raised region extend along substantially the entire length of the central portion. The recessed region receives the strap of the mask.

In one embodiment, the cheek pads having a width W which is greater than or equal to the width of the central portion. The cheek pads serve as padding or protection for the corners of the mask when the mask and protective garment are worn by the patient.

In one embodiment, the protective garment is made from a knitted material, such as for example a four-ply hypoallergenic acrylic yarn. In one embodiment, the knitted material is knitted to a double thickness (two layers of knitted material). In another embodiment, the knitted material is knitted to a single thickness. The protective garment can be made from other soft, resilient materials, such as a foam material. For example, the garment could be stamped or formed from a blank of foam material to have the cheek pads, central portion with recessed portion and peripheral raised portions.

In one embodiment, the mask protector is essentially double sided, in that it has a front side and a opposite rear side. The central portion of mask protector is provided with a recessed region and peripheral raised regions in both the front side and the opposite rear side, with the recessed region and peripheral raised regions extending substantially the entire length of the central portion. In this embodiment, the nurse or person fitting the mask protector to the patient need not closely inspect the mask protector to see which side of the protector has the channel in the central portion in order to properly fit the mask strap into the channel.

The mask may further include a hole in each of the cheek pads. The hole facilitates retaining the mask protector to the mask and strap in the manner described in more detail below.

The mask protector can be made in sizes to fit infants, youth, adults and the elderly and may be made in a variety of sizes. When flat and in an unstretched condition, the mask protector has an overall length of between 10 and 25 inches.

In another aspect, a method is disclosed of making a mask protector adapted to be worn by a patient fitted with mask having a strap. The method includes the step of knitting a protective garment in the form of a single piece of soft, relatively thick and resilient material. The garment includes (1) an elongate central portion having a first end and an opposite second end, the central portion having a recessed region and a peripheral raised region, the recessed region and peripheral raised region extending substantially the entire length of the central portion, the recessed region for receiving the strap of the mask, and (2) a first cheek pad integral with the first end of the central portion and a second cheek pad integral with the second end of the central portion.

In one embodiment, the first and second cheek pads having a width W greater than or equal to the width of the relatively narrow central portion.

In still another aspect, a medical apparatus assembly is disclosed comprising in combination: a mask (e.g., oxygen mask) having a peripheral region including features such as buckle-type fittings or slots for fitting an elastic strap to the mask, and a protector for the mask comprising a protective garment constructed from a soft, resilient material. The protective garment includes (1) an elongate central portion having a first end and an opposite second end, the central portion having a recessed region and a peripheral raised region, the recessed region and peripheral raised region extending substantially the entire length of the central portion, the recessed region for receiving the strap of the mask, and (2) a first cheek pad integral with the first end of the central portion and a second cheek pad integral with the second end of the central portion. The cheek pads provide a protective cover for the features for fitting the elastic strap for the mask.

The material may take the form of a knitted or foam material. For example the material may take the form of a four-ply hypoallergenic acrylic yarn, knitted to a single or a double thickness. The cheek pads may further include a hole. The central portion may also have a channel or recessed portion and adjacent raised portions in both the front side and the back side of the central portion, making the protective garment essentially double-sided.

### BRIEF DESCRIPTION OF THE DRAWINGS

FIG. 1 is a top view of a first embodiment of a protective garment for masks such as oxygen masks used by patients.

FIG. 2 is a top view of a second embodiment of a protective garment for masks.

FIG. 3 is a cross-section of the embodiment of FIG. 2 along the lines 3-3 of FIG. 2.

FIG. 4 is a cross-section of the embodiment of FIG. 1 along the lines 4-4 of FIG. 1.

FIG. 5 is an illustration of a patient wearing an oxygen mask and the oxygen mask protector of this invention.

FIG. 6 is a view showing the side of the patient's head of FIG. 5, showing the protection of the ears, cheek and back of the head by virtue of the features of the mask protector of this invention.

FIG. 7 is a more detailed close-up view of the mask strap and mask protector of FIG. 6.

FIG. 8 is a view showing the rear of the patient's head showing the protection provided by the mask protector of this invention.

FIG. 9 is a perspective view of the mask and strap of FIGS. 5-8 removed from the patient. The channel in the mask protector facilitates removal and installation of the mask and mask protector as a unit.

FIG. 10 is a more detailed view of the peripheral portion of the mask of FIG. 4 showing the features in the mask for receiving the mask strap.

### DETAILED DESCRIPTION

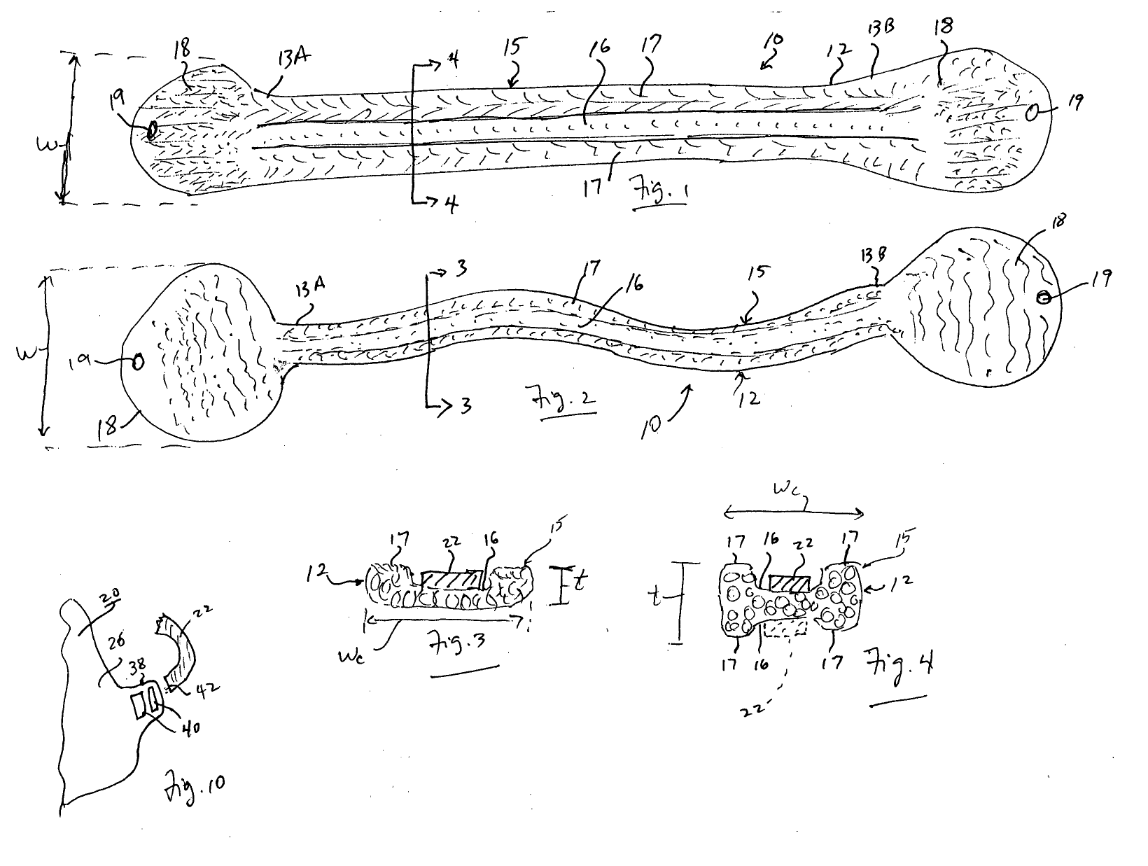

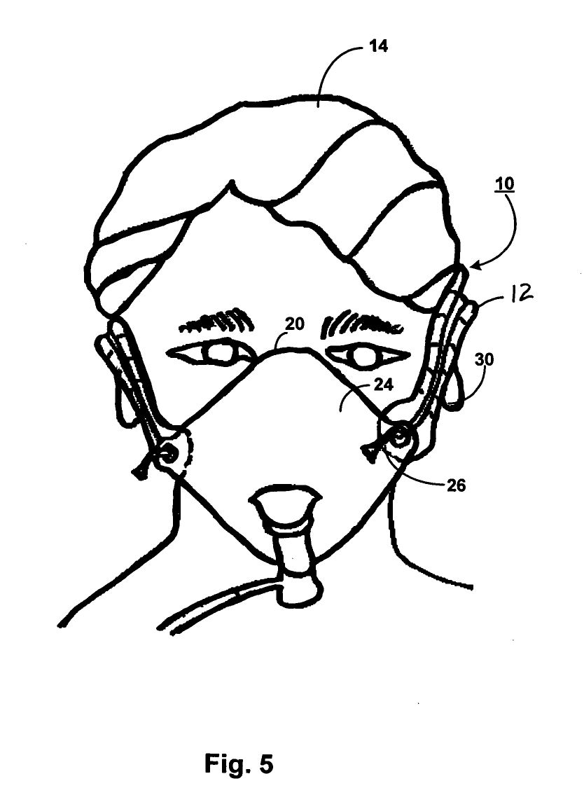

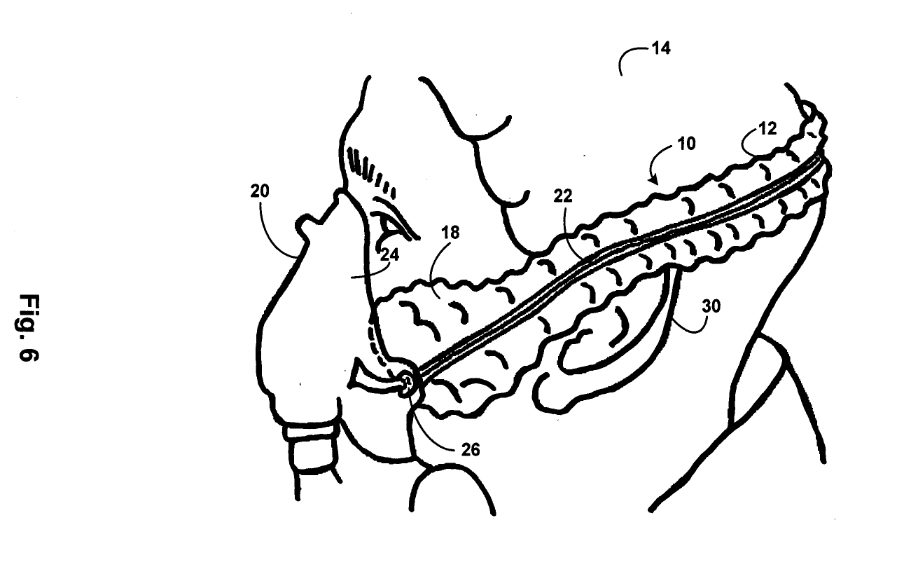

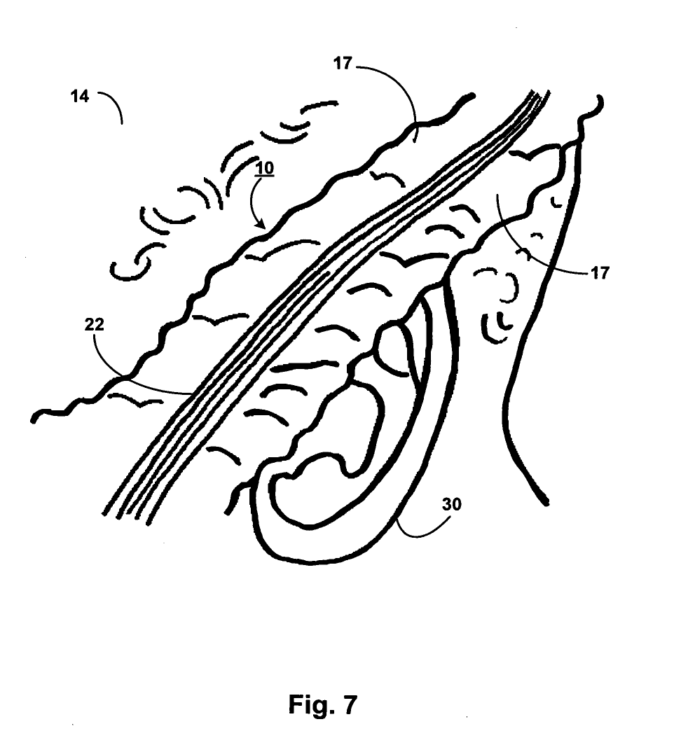

FIG. 1 is a top view of a first embodiment of a mask protective garment 10 in accordance with the invention. The garment 10 is adapted to be worn by a patient 14 who is fitted with and wearing a mask 20 having a strap 22, as illustrated in FIGS. 5-8 and as described in detail below. The mask 20 may be an oxygen mask or other type of mask, and the particulars of its construction are not important. FIG. 4 is a cross-section of the embodiment of FIG. 1 along the lines 4-4 of FIG. 1.

With reference to FIGS. 1, 4 and 5-8, the mask protector is in the form of a protective garment 10 which constructed from a soft, resilient material 12. The material is preferably a hypoallergenic material, and may be sterilized or sanitized as appropriate for wearing on a patient. The garment may be made from a knitted material, such as an acrylic yarn (preferably four-ply hypoallergenic acrylic yarn), or may be made from a non-knitted material such as foam material either with or without a covering or backing. While FIGS. 1-8 show a knitted garment 10, this is provided by way of example and not limitation.

The mask protector garment 10 has a particular shape and configuration which is designed to protect the patient from possible chafing from wearing a mask. The sources of chafing are the mask strap 22 and the mask 20 itself. To provide such protection, and with reference to FIG. 1, the garment 10 is in the form of an elongate, relatively narrow central portion 15, typically between 10 and 20 inches in length, having a first end 13A and an opposite second end 14B, and cheek pads 18 integral with the ends 13A, 13B of the central portion 15. The central portion 15 has a recessed region 16 and peripheral, adjacent raised regions 17 extending the length of the central portion. The recessed region 16 provides a channel or slot in the central portion 15 for receiving the strap 22 of the mask, as best shown in FIGS. 4, 6, 7, 8 and 9. The peripheral raised regions 17 define the channel or slot 16, preventing the mask strap 20 from coming out of the slot 16, and serves as additional padding for protection of the ears 30 where the mask strap covers the tops of the ears, as best shown in FIGS. 5-8.

The embodiment of FIG. 1 is double sided (having identical front and back sides), in that both front and back sides of the central portion 15 have the central channel or slot 16 running the length thereof. Thus, while FIG. 1 shows one side of the garment 10, the opposite side is identical in construction and configuration. In this embodiment, the nurse or other person attending to the patient does not have to closely inspect the garment 10 to determine which side has the channel 16, as it is present in both sides. The representative embodiment of FIG. 1 is designed for an adult patient, has an overall length of between about 15 and 20 inches, has a thickness T (FIG. 4) of about ½ inch (preferably between ⅛ and ¾ inch), and a width of the central portion (W_{c}, FIG. 4) of approximately 1¼ inch. The width of the channel 16 is between ¼ and ½ inches, but this may vary depending on the width of the mask strap 22.

The width W of the cheek pads (FIG. 1) can vary but is preferably greater than the width W_{c} (FIG. 4) of the central portion 15, in order to provide adequate surface area to reliably protect the cheeks and adjacent portions of the face from chafing from the peripheral edge of the mask 22. In the preferred embodiments, the cheek pads 18 are roughly circular in shape and have a diameter of between about 2 and about 3 inches (W is between about 2 and 3 inches). The Thickness of the cheek pads can be less than the thickness T of the central portion, for example a thickness of between ⅛ to ⅜ inches.

In one possible embodiment, the cheek pads 18 have a hole 19 formed in the cheek pad. When the garment 10 is being prepared to be installed on the patient's head and face, and with reference to FIG. 10, the ends 42 of the mask strap 22 are inserted through the holes 19 and then through the features 44 (e.g., slot, hole or slots) in the peripheral portion 26 of the mask which are designed to enable the ends of the mask strap 22 to be fastened the mask 20. Some masks have a tab 38 in the peripheral portion of the mask which includes a pair of slots 44. The holes 19 in the cheek pads are positioned such that when the mask strap 22 is threaded through the hole 19 and fastened to the features 40, the mask strap 22 essentially holds the garment 10 to the mask 20 and strap 22, allowing the mask 20 and garment 10 to be removed from and installed onto the patient 14 as a unit.

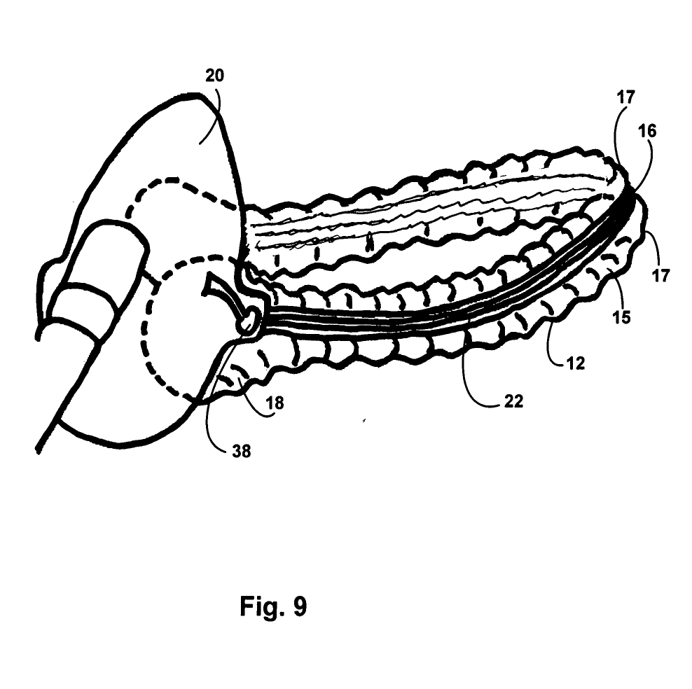

The nesting of the strap 22 within the channel 16 further helps secure the strap 22 to the garment 10. For example, as shown in FIG. 9, the strap and garment 10 are fitted to the mask 20 and then the entire assembly of mask 20, strap 22 and garment 10 are ready to fit onto the head and face of the patient, resulting in the protection of the head and face as shown in FIGS. 5-8. Even if the cheek pads 18 do not have the hole 19, the strap 22 is nested within the channel 16 and this is typically sufficient to allow the mask 20, strap 22 and garment 10 to be installed onto the patient and removed from the patient as a unit.

In FIG. 1, the embodiment is shown knitted as a double layer of four ply, hypoallergenic acrylic yarn. Double-layer knitting tends to prevent the garment from taking on a curl or set, which can occur if the garment is knitted as a single layer of material.

FIG. 2 shows an embodiment in which the garment 10 is knit from a single layer of material 12. The peripheral raised portions 17 are achieved by additional knitting in these regions to define the raised portion 17 and the relatively thinner slot or channel 16. As shown in the cross-section of FIG. 3, the single layer embodiment has only one side with the channel 16, making this embodiment having a front side (shown in FIG. 2) with a channel. The back side (opposite side from the side shown in FIG. 2) does not have the channel, as can be ascertained from FIG. 3. The embodiment of FIG. 2 has an overall length from end to end of about 20 inches, a thickness T in the central portion of about ¼ inch, and a thickness in the cheek pad of about ¼ inch. The width W in FIG. 2 is about 2 inches in the illustrated embodiment.

As noted previously, the mask protector 10 can be manufactured in a variety of sizes to fit infants, young adults, adults and the elderly. The overall end-to-end length of the mask protector garment 10, when laid flat and in an unstretched condition will be between about 10 and about 25 inches. For mask protectors 10 which are designed to be worn by children, the masks may decorated in bright colors and patterns, and decorated with clowns, characters, or other styling touches.

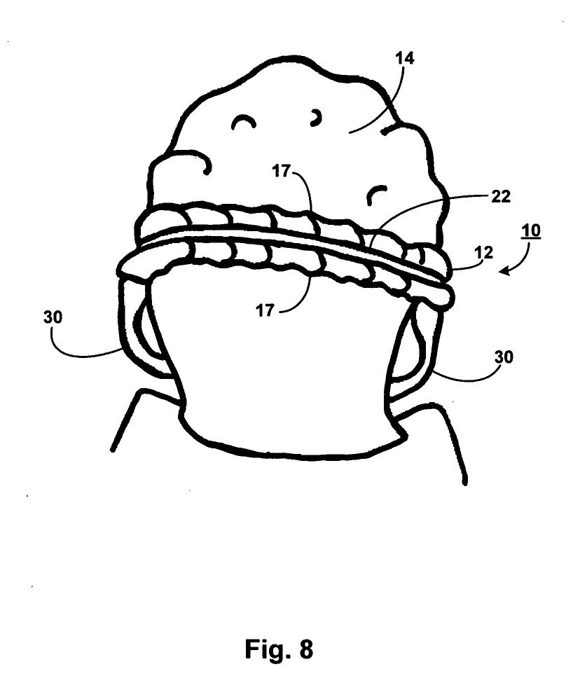

Referring now in particular to FIGS. 5-8, when the mask 20 is worn by a patient 14, without the protection features provided by the garment of this invention, the mask itself and the strap are in direct contact with the skin of the patient and, over time, cause the skin to chafe. Open sores can develop, which providing a site for possible bacterial infection. This is a particularly acute problem with some patients, including those that have suffered trauma or burns to the face, the elderly, critically ill patients, and patients with depressed immune systems. However, with the protection provided by the mask protector 10 of this invention, these chafe and potential infection problems are minimized. As illustrated in FIGS. 5-8, the peripheral portions 24 and 26 of the mask in the region of the cheek bones are not in contact with the skin of the patient due to the presence of the cheek pads 18 providing padding over the rim of the mask 20 in this area. Furthermore, the strap 22 does not touch the patient's skin at all. The garment provides a padding for the strap 22 from one end of the strap to another, particularly over the tops of the ears 30 as shown in FIGS. 5, 6, 7 and 8. The garment further protects the back of the head, as shown in FIG. 8. Additionally, the garment prevents the strap from getting tangled in, or pulling on, the patient's hair when the mask strap 22 is installed, adjusted, or removed from the patient.

Manufacturing Considerations

From the above discussion, it will be appreciated that I have also disclosed a method of making a mask protector 10 adapted to be worn by a patient 14 fitted with mask 20 having a strap 14, comprising: knitting a protective garment 10 in the form of a single piece of soft, relatively thick and resilient material, the garment comprising (1) an elongate central portion 15 having a first end 13A and an opposite second end 13B, the central portion having a recessed region and a peripheral raised region, the recessed region and peripheral raised region extending substantially the entire length of the central portion, the recessed region for receiving the strap of the mask, and (2) a first cheek pad integral with the first end of the central portion and a second cheek pad integral with the second end of the central portion.

In one embodiment, as shown in FIGS. 1 and 2, the first and second cheek pads 18 having a width W greater than or equal to the width W_{c} of the relatively narrow central portion.

In one embodiment, the knitted material comprises a four-ply hypoallergenic acrylic yarn. In one embodiment, the knitted material is knitted to a double thickness. In other embodiments, the knitted material is knitted to a single thickness.

The garment 10 can also be made through other means as will be understood in the garment arts besides knitting. For example, it can be stamped from a solid piece of material (e.g. foam) and the channel 16 provided in the foam by means of a die or other process. It could also be formed in a mold with a rubber or plastic material, and then optionally fitted with a cover made from a comfortable material such as cotton or other material.

In one variation, the material for the protective garment 10 is made from a light, stretchy material, which may be a cotton, foam-like, or gauze-like material, or other soft material such as yarn, preferably hypoallergenic. A further example is a stretchy “memory” yard which stretches but contracts to its original un-stretched condition when stretching forces are removed.

Embodiments in which the material is of double thickness in the central portion 15 are a preferred embodiment (basically, the central portion is a flattened tube). In these embodiments, a space exists between the two layers of material (upper and lower layers) and the strap for the mask can be threaded through the hole in one of the cheek pads and passed between the layers of the central portion and out of the hole in the other check pad. In these embodiments, the strap for the mask is completely contained within the central portion 15 of the garment 10, ensuring that the mask does not cause chafing of the patient's skin.

The garment is preferably UV sterilized and packaged prior to shipment to the customer (e.g., hospital.) Ideally, the UV sterilization occurs in the package itself. In this embodiment, the material should be to relatively light-weight material such as 2-ply yarn or foam.

Mask and Protector Combination

It will further be appreciated that I have described a medical apparatus assembly comprising, in combination: a mask 20 having a peripheral region 24, 26 including features (e.g., slots 40 or a hole) for fitting an elastic strap 22 to the mask 20; and a protector 10 for the mask comprising a protective garment 10 constructed from a soft, resilient material and comprising (1) an elongate central portion 15 having a first end 13A and an opposite second end 13B, the central portion having a recessed region 16 and a peripheral raised region 16, the recessed region and peripheral raised region extending substantially the entire length of the central portion (as shown in FIGS. 1, 2 and 5-8), the recessed region for receiving the strap 22 of the mask 20, and (2) a first cheek pad 18 integral with the first end of the central portion 15 and a second cheek pad 18 integral with the second end of the central portion.

The first and second cheek pads having a width W greater than or equal to the width W_{c} of the central portion 15. The cheek pads 18 provide a protective cover for the features 40 for fitting or attaching the elastic strap to the mask.

As noted above, the material may take the form of a knitted material such as a four-ply hypoallergenic acrylic yarn, in either a single or double thickness. The material may also be foam or other material, optionally covered with a cover made from a comfortable fabric.

In preferred embodiments, the mask protector 10 has a front side and an opposite rear side. The central portion 15 is provided with a recessed region and a peripheral raised region in both the front side and the opposite rear side (as shown in FIG. 4 and described above), with the recessed region 16 and peripheral raised region 17 of the front side and the opposite rear side extending substantially the entire length of the central portion 15.

Variation from the specifics of the above-described embodiments is of course possible without departure from the scope of the invention. All questions concerning scope are to be determined by reference to the appended claims.

1. A mask protector adapted to be worn by a patient fitted with mask having a strap, comprising:
a protective garment constructed from a soft, resilient material and comprising (1) an elongate central portion having a first end and an opposite second end, the central portion having a recessed region and adjacent peripheral raised regions, the recessed region and peripheral raised regions extending substantially the entire length of the central portion, the recessed region adapted for receiving the strap of the mask, and (2) a first cheek pad integral with the first end of the central portion and a second cheek pad integral with the second end of the central portion.

2. The mask protector of claim 1, wherein the first and second cheek pads have a width W greater than or equal to a width W_{c} of the central portion.

3. The mask protector of claim 1, wherein the material comprises a knitted material.

4. The mask protector of claim 3, wherein the knitted material comprises a hypoallergenic acrylic yarn.

5. The mask protector of claim 1, wherein the material has a double thickness in the central portion.

6. The mask protector of claim 3, wherein the knitted material comprises a four-ply acrylic yarn.

7. The mask protector of claim 2, wherein each of the cheek pads further includes a hole.

8. The mask protector of claim 1, wherein the material comprises a foam material.

9. The mask protector of claim 1, wherein the mask protector has a front side and a opposite rear side, and wherein the central portion is provided with a recessed region and adjacent peripheral raised regions in both the front side and the opposite rear side, the recessed region and peripheral raised regions of the front side and the opposite rear side extending substantially the entire length of the central portion.

10. The mask protector of claim 1, wherein the overall length of the mask protector, when laid flat and in an unstretched condition, is between 10 and 25 inches.

11. A method of making a mask protector adapted to be worn by a patient fitted with mask having a strap, comprising:
knitting a protective garment in the form of a single piece of soft, relatively thick and resilient material, the garment comprising (1) an elongate central portion having a first end and an opposite second end, the central portion having a recessed region and a peripheral raised region, the recessed region and peripheral raised region extending substantially the entire length of the central portion, the recessed region for receiving the strap of the mask, and (2) a first cheek pad integral with the first end of the central portion and a second cheek pad integral with the second end of the central portion.

12. The method of claim 11, wherein the knitted material comprises a four-ply hypoallergenic acrylic yarn.

13. The method of claim 11, wherein the knitted material is knitted to a double thickness.

14. A medical apparatus assembly comprising, in combination:
a mask having a peripheral region including features for fitting an elastic strap to the mask:
a protector for the mask comprising a protective garment constructed from a soft, resilient material and comprising (1) an elongate central portion having a first end and an opposite second end, the central portion having a recessed region and a peripheral adjacent raised regions, the recessed region and peripheral raised regions extending substantially the entire length of the central portion, the recessed region for receiving the strap of the mask, and (2) a first cheek pad integral with the first end of the central portion and a second cheek pad integral with the second end of the central portion.

15. The medical apparatus assembly of claim 14. wherein the first and second cheek pads having a width W greater than or equal to a width W_{c} of the central portion, the first and second cheek pads providing a protective padding for the mask in the peripheral region of the mask.

16. The medical apparatus assembly of claim 14, wherein the material comprises a knitted material.

17. The medical apparatus assembly of claim 16, wherein the knitted material comprises a four-ply hypoallergenic acrylic yarn.

18. The medical apparatus assembly of claim 14, wherein the material comprises a double thickness material.

19. The medical apparatus assembly of claim 15, wherein each of the cheek pads further include a hole.

20. The medical apparatus assembly of claim 14, wherein the material comprises a foam material.

21. The medical apparatus assembly of claim 14, wherein the mask protector has a front side and a opposite rear side, and wherein the central portion is provided with a recessed region and adjacent peripheral raised regions in both the front side and the opposite rear side, the recessed region and peripheral raised regions of the front side and the opposite rear side extending substantially the entire length of the central portion.
